(19) United States
(12) Patent Application Publication
(10) Pub. No.: US 2019/0336626 A1
(43) Pub. Date: Nov. 7, 2019

(54) Attachable Photocatalytic Device

(71) Applicant: Aleddra Inc., Renton, WA (US)

(72) Inventors: Chia-Yiu Maa, Bellevue, WA (US); Chun-Te Yu, Bellevue, WA (US)

(21) Appl. No.: 15/969,987

(22) Filed: May 3, 2018

(51) Int. Cl. A61L 2/08; A61L 2/10

(52) U.S. Cl. CPC A61L 2/088; A61L 2/10

(57) ABSTRACT

A photocatalytic device includes one transparent or translucent carrier and at least one photocatalytic film. The carrier is in the form of a sheet with two opposite surface. At least one of the two surfaces of the carrier is at least partially coated with the photocatalytic film. The photocatalytic film is photocatalytic activated by visible light with wavelength>400 nm for providing the antibacterial functionality. In some situation, only the outer surface of the carrier is coated with the photocatalytic film. In other situation, only the inner surface of the carrier is coated with the photocatalytic film. There are situations where both surfaces of the carrier are coated with the photocatalytic film. The carrier has an attaching mechanism for attaching the carrier to the external solid object, and the carrier is attachable to and detachable from the external solid object hands of a user without using a tool.

### BACKGROUND

### Technical Field

The present disclosure pertains to the field of antibacterial photocatalytic devices and, more specifically, proposes an attachable antibacterial photocatalytic device.

### Description of Related Art

Presently there is a technique that advocates the use of a photocatalytic film coated on a carrier in which the photocatalytic film comprises rhombus-shape anatase-type titanium dioxide (TiO₂). When exposed and excited by a suitable light source, the photocatalytic film generates radicals on its surface that are effective in killing and inhibiting the bacteria in making contact with the photocatalytic film. There are two means where the bacteria may connect with the photocatalytic film. Firstly, the airborne bacteria may be brought to the surface of the photocatalytic film due to the air circulation. Secondly, a bacteria carrier such as a person may make physical contact with the surface of the photocatalytic film.

There are numerous constraints with this prior art technique of applying the photocatalytic film directly onto the carrier. Firstly, the coating of the photocatalytic film on the barrier is non-removable. This may be suitable for some applications. However, it is undesirable for application where the photocatalytic film needs to be removed before its final use. Secondly, the antibacterial effect depends on the availability of the photocatalytic film on the carrier. If the photocatalytic film is outwearing due to scrubbing, scratching, or simply the normal wear-and-tear, then the antibacterial effect would disappear along with the wearing out of the photocatalytic film. Thirdly, the effectiveness of the photocatalytic film depends highly on the nature and thus the integrity of the crystal structure of the titanium dioxide. As a result, useful functionality, such as blue light filtering (for protection of the eyes), scratch resistance, shatter-resistance, or heat insulation, may not be added to the photocatalytic film without affecting the integrity of the crystal structure of the titanium dioxide and the effectiveness of the antibacterial function of the photocatalytic film. This issue is not addressed by the prior art technique.

The prior art technique focuses on the structure of titanium dioxide based photocatalytic film and the addition of silica or nano silvers to the titanium dioxide. The prior art technique also provides a plating process to ensure a strong binding of the photocatalytic film over the carrier. The prior art technique did not address implicitly or explicitly the three limitations of coating the titanium dioxide based photocatalytic film directly on the carrier as mentioned above. Firstly, the coated photocatalytic film is non-removable, secondly, some portion of the photocatalytic coasting may be worn off, and thirdly new useful functionality cannot be added to the photocatalytic film without negatively impacting the crystal structure of the titanium dioxide and lessening the antibacterial effect of the photocatalytic film.

The present disclosure presents an attachable antibacterial photocatalytic device where it could be easily attached to or detached from an external solid object, thus overcoming the three limitations of the prior art technique without sacrificing its antibacterial effectiveness.

### SUMMARY

In one aspect, the photocatalytic device comprises one transparent or translucent carrier and at least one photocatalytic film. The carrier is in the form of a sheet with two opposite surfaces. At least one of the two surfaces of the carrier is at least partially coated with the photocatalytic film. The photocatalytic film may be photocatalytic activated by visible light with wavelength>400 nm for providing antibacterial protection. UV light is not required for activating the photocatalytic film in the present disclosure. The carrier has an attaching mechanism for attaching the carrier to an external solid object. The carrier is attachable to, and detachable from the external solid object with hands only, using no tools.

The key difference with the present disclosure from the prior art technique is that the present disclosure has a tool-free attaching and detaching mechanism, which enhances greatly the usability of the photocatalytic structure taught by the prior art technique. Moreover, in some situations, only the outer surface of the carrier, that is the surface not making contact with the external solid object, is coated with the photocatalytic film. In other situations, only the inner surface of the carrier, that is the surface making contact with the external solid object, is coated with the photocatalytic film. There may be situations where both surfaces of the carrier are coated with the photocatalytic film. It is not required to coat the complete surface(s) of the carrier with the photocatalytic film. Rather, only the surface area of the carrier that needs antibacterial protection is coated with the photocatalytic film. Furthermore, the carrier is not permanently adhered or attached to the external solid object. With its tool-free attaching mechanism, the carrier can be easily attached to and removed from the external solid object by hands only, using no tools. The carrier is transparent or translucent as to allow the light to shine through the carrier, thus activating the photocatalytic film, irrespective of which surface(s) of the carrier is coated with the photocatalytic film.

With the present disclosure, the first limitation of the prior art technique on a non-removable photocatalytic film is overcome, since the carrier may be easily detached from the external solid object. The second limitation of the prior art technique on the wearing off of the photocatalytic film is also overcome by replacing a worn-off carrier with a new carrier. The third limitation of the prior art technique on not being able to add useful functionality to the photocatalytic film can also be resolved by adding the new functionality to the carrier (material) rather than the photocatalytic film.

In some embodiments, the carrier may be made of flexible material such as plastics or rigid material such as glass. When the carrier is made of flexible material, the carrier may be able to attach to the external solid object with non-even surfaces. One example is a plastic food wrap. The wrap carrier is coated on the inside surface with a photocatalytic film. This plastic food wrap is able to wrap over non-even surfaces easily, adding the antibacterial protection over the items wrapped.

In some embodiments, the photocatalytic film coated on the surface(s) of the carrier may contain rhombus-shape anatase-type titanium dioxide (TiO₂). As taught by the prior art technique, some active ingredient(s) such as silica and nano silver particles may be added to the rhombus-shape anatase-type titanium dioxide for improving the antibacterial effectiveness. The present disclosure, however, is not limited to using only rhombus-shape anatase-type titanium dioxide, nor to of silica and nano silvers as the only additive options to titanium dioxide.

In some embodiments, the carrier may be tinted to filter light in certain or predetermined wavelength ranges. For example, when the carrier is in the form of a screen protector and the external solid object is the screen of a touchscreen display (of a computer, tablet computer, or cellphone), then it would be useful to have the carrier tinted for filtering out blue light, which is known to be harmful to human eyes. The carrier continues to allow the light in other wavelength ranges to pass through, thus still activating the photocatalytic film coated on the surface(s) of the carrier, which is in the form of a screen protector.

In some embodiments the carrier may be scratch-resistant, and in other embodiments the carrier may be shatter-resistant. It is very likely that the carrier in the form of a screen protector for a touchscreen display may be both scratch-resistant and shatter-resistant.

In some embodiments, the carrier serves as a heat insulator. When the carrier is in the form of a window film for attaching over a window, it may be desirable to use heat insulating material for constructing the carrier. Heat-insulating window film would add heat insulation to a home and lower the energy consumption for air-conditioning in the summer and for heating equipment in the winter.

In some embodiments, the carrier comprises a solar cell. Transparent solar cells have been introduced recently with acceptable electricity generation efficiency while allowing most light to pass through. As a result a transparent solar cell may be used as the carrier of the present disclosure.

In some embodiments, the attaching mechanism for attaching the carrier to the external solid object may be an adhesive at least partially coated on one of the two surfaces of the carrier for adhering this surface of the carrier to the external solid object. When the carrier is in the form of a touchscreen protector, the adhesive coating is likely to be added to the inside the surface of the carrier. The coated adhesive would then attach the carrier to the touchscreen display. For such an application, a weak (non-stick) adhesive is often used so the carrier (the touchscreen protector) can be easily detached from the external solid object (the touchscreen display).

In some embodiments, the attaching mechanism for attaching the carrier to the external solid object may be the electrostatics between the carrier and the external solid object. When the carrier is made as a window film, it is attachable to the external solid object (the window) via electrostatics. In such case, the photocatalytic film is coated on the outside surface of the carrier, which is the surface that is not making contact with the window.

In some embodiments, the attaching mechanism for attaching the carrier to the external solid object may be the weight of the carrier. In the application where the carrier is made as a keyboard protector, the weight of the carrier secures it to the external solid object, the computer keyboard. Additional mechanism, such as the matching pattern of the carrier with the pattern of the keyboard reinforces the carrier (the keyboard protector) from changing positions. Nevertheless, it is the weight of the carrier that keeps it on the keyboard.

In some embodiments, the attaching mechanism for attaching the carrier to the external solid object may be an elastic wrap around the edge of the carrier over the external solid object. The elastic wrap has a firm grasp of the external solid object and may be easily attached to and detached from the external solid object.

In some embodiments, the attaching mechanism for attaching the carrier to the external solid object may be a Velcro between the carrier and the external solid object.

In some embodiments, the attaching mechanism for attaching the carrier to the external solid object may be any physical or mechanical locking mechanism locking the carrier to the external solid object by hands, using no tools.

In some embodiments, the attaching mechanism for attaching the carrier to the external solid object may be the wrapping of the carrier over the external solid object. In the application where the carrier is made as a plastic food wrapper, the photocatalytic film is coated on the inside surface of carrier and the external solid object is a food item. When the carrier (the plastic food wrap) is wrapped around the external solid object (the food item), the photocatalytic film is making contact with the food item and its antibacterial function will kill the bacteria or inhibit the growth of mold on the surface of the external solid object (the food item). The rhombus-shape anatase-type titanium dioxide is non-chemical, odorless, and most importantly non-toxic. It is thus a good candidate for using it against bacteria and mold growth on a food item. When coating the titanium dioxide on the inside surface of the plastic food wrap (the carrier), the wrap can be easily removed before the consumption of the food item.

In some embodiments, the attaching mechanism for attaching the carrier to the external solid object may be having the carrier in the form of a bag for bagging the external solid object. One example is that the carrier is in the form of a zipper bag where the photocatalytic film is coated in the inside surface of the zipper bag. In this case the photocatalytic film would kill the bacteria or inhibit the growth of mold on the surface of the external solid object (the food item) stored inside the bag.

### BRIEF DESCRIPTION OF THE DRAWINGS

The accompanying drawings are included to aid further understanding of the present disclosure, and are incorporated in and constitute a part of the present disclosure. The drawings illustrate a select number of embodiments of the present disclosure and, together with the detailed description below, serve to explain the principles of the present disclosure. It is appreciable that the drawings are not necessarily to scale, as some components may be shown to be out of proportion to size in actual implementation in order to clearly illustrate the concept of the present disclosure.

FIG. 1 schematically depicts a diagram of a photocatalytic device where the external solid object is a window and the attachable carrier is a window film.

FIG. 2 schematically depicts a diagram of a photocatalytic device where the external solid object is a touchscreen display and the attachable carrier is a screen protector.

FIG. 3 schematically depicts a diagram of a photocatalytic device where the external solid object is a computer keyboard and the attachable carrier is a keyboard protector with a locking mechanism.

FIG. 4 schematically depicts a diagram of a photocatalytic device where the external solid object is a food item and the attachable carrier is plastic food wrap coated with photocatalytic film.

### DETAILED DESCRIPTION OF PREFERRED EMBODIMENTS

### Overview

Various implementations of the present disclosure and related inventive concepts are described below. It should be acknowledged, however, that the present disclosure is not limited to any particular manner of implementation, and that the various embodiments discussed explicitly herein are primarily for purposes of illustration. For example, the various concepts discussed herein may be suitably implemented in a variety of photocatalytic devices having different form factors.

The present disclosure discloses a photocatalytic device that one transparent or translucent carrier and a photocatalytic film. The carrier is in the form of a sheet with two surfaces At least one of the two surfaces of the carrier is at least partially coated with the photocatalytic film. The carrier has an attaching mechanism for attaching the carrier to an external solid object, and the carrier is attachable to and detachable from the external solid object with hands only, using no tools.

### Example Implementations

The FIG. 1 is an embodiment of the photocatalytic device of the present disclosure where the attachable carrier 101 is in the form of a laminate window film and the external solid object is a window 102. The carrier 101 is made of polyethylene terephthalate (PET) and provides additional heat-insulation to the window 102. The outside surface of the carrier is coated with titanium dioxide film 103. There is no adhesive used on the inside surface of the carrier 101. The carrier is attached onto the window on its inside surface via electrostatics. By leveraging the large surface area of the window, this carrier provides a large photocatalytic active surface for killing any airborne microbial making contact to the titanium dioxide film on the carrier. This offers an effective means of antibacterial/antiviral protection for any rooms with large windows, such as classrooms, offices with large windows, and most residential rooms. The sunlight and the regular lighting fixtures or light bulbs all contain suitable visible light with wavelength>400 nm to activate the photocatalytic film coated on the carrier. UV light is not required. When the photocatalytic film on the carrier is worn off, the entire carrier (the window film) can then be easily detached and a new window film be attached.

It may be argued that the window film application above is obvious in view of the prior art technique. By tracing the history, photocatalyst has been introduced since 1996, and the window film has been used for decades as a winterization treatment on single-layer windows in cold weather zones. If it had been so obvious, then a photocatalyst coated window film would have been invented within the last 20 years. It is not the case. Therefore the novelty and the non-obviousness of the present disclosure as applied to the window film application is validated. Another key difference between the present disclosure and the prior art technique is that the prior art technique teaches to coat the photocatalytic film on the carrier, not under the carrier. As such, when the light shines on the device according to the prior art technique, the light shines through the photocatalytic film first before reaching the carrier. With the present disclosure, the light shines through the external solid object (the window) first, then through the carrier (the window film), and then finally through the photocatalytic film. This application is not taught by the prior art technique implicitly or explicitly.

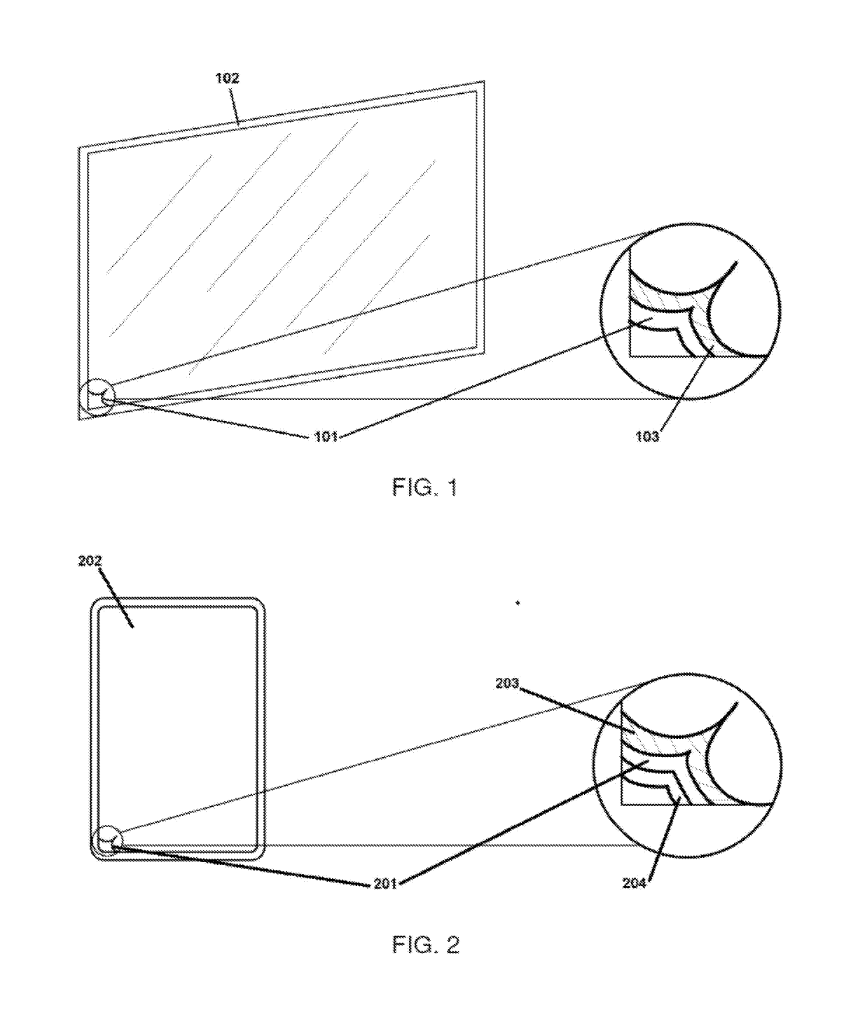

The FIG. 2 is an embodiment of the photocatalytic device of the present disclosure where the carrier is in the form of a screen protector 201 and the external solid object is the touchscreen of a cellphone 202. The carrier is made of tempered glass for scratch-resistant and shatter-resistant. It is also tinted to filter out blue light for eye protection. The outside surface of the carrier is coated with titanium dioxide film 203, and the inside surface of the carrier is coated with an adhesive 204 for attaching the carrier to the cellphone screen. The light generated by the cellphone touchscreen provides suitable visible light with wavelength>400 nm to activate the photocatalytic film. No UV light is required. Rather than killing the airborne bacteria as shown in the window film application previously mentioned, the photocatalyst coated screen protector aims at killing the bacteria and the virus left by the user(s) when making contact on the screen protector with their fingers, or when it comes in contact with unsanitary surfaces, such as desks, bathrooms, public seating, counters. This prevents the transmission of diseases via a shared touchscreen by multiple users or via unsanitary surfaces. When the photocatalytic film is worn off due to heavy use, the user can easily replace it with a new photocatalyst coated screen protector.

A removable screen protector was first introduced by Herbert et al. in U.S. Pat. No. 3,418,426A in 1962 for TV screen. As mentioned previously, the photocatalyst has been introduced since 1996 by Soma et al. in U.S. Pat. No. 6,242,752B1 in 1996. Has it been obvious of making photocatalyst coated screen protector, there should be many such products, and there is none. Therefore the novelty and the non-obviousness of the present disclosure as applied to the touchscreen protector application is validated. Another key difference between the present disclosure and the prior art technique is that the prior art technique teaches to coat the photocatalytic film on of the carrier, not under the carrier. As such, when the light shines on the device, the light shines through the photocatalytic film first before reaching the carrier. With the present disclosure, the light shines through the external solid object (the touchscreen panel) first, then through the carrier (the screen protector), and then finally through the photocatalytic film. This application is not taught by the prior art technique implicitly or explicitly.

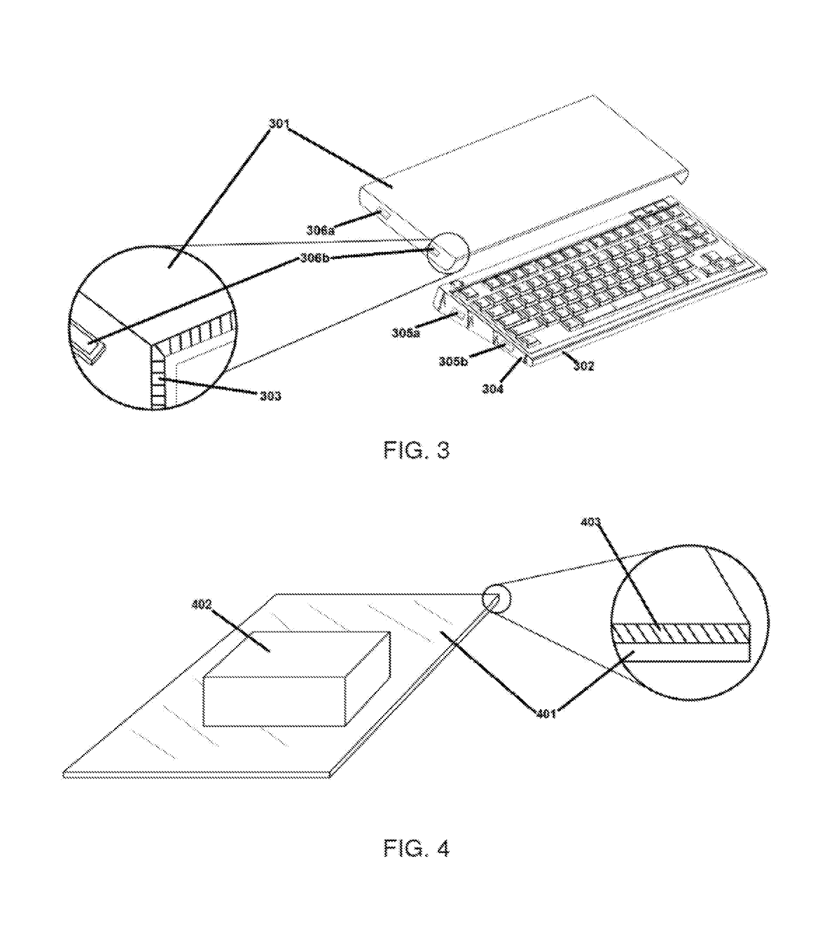

The FIG. 3 is an embodiment of the photocatalytic device of the present disclosure where the carrier is in the form of a keyboard protector 301 and the external solid object is a computer keyboard 302. The carrier is made of plastic material. The photocatalytic film 303 is coated on the top surface of the carrier. On each of the two short sides of the keyboard 304, there are two holes 305a and 305b. There are two knots (306a and 306b) on the inside of each of the two short edges of the carrier. The carrier is attached to the keyboard by pressing the two knots into the two holes of the keyboard. Other attaching mechanism such as an elastic wrap around the edge of the carrier, or a Velcro between the carrier and the keyboard, can also be used for attaching the carrier to the keyboard. Having the photocatalyst coated on the top surface of the carrier can inhibit effectively the transmission of the infectious diseases via a shared keyboard in the environment such as classrooms, public libraries, and hospitals. The photocatalytic film coated on the carrier may be photocatalytic activated by visible light with wavelength>400 nm. No UV light is required. When the photocatalytic film is worn off, the keyboard protector can be easily replaced with a new one. the prior art technique does not suggest implicitly or explicitly a physical or mechanical attaching mechanism for attaching his photocatalytic device to an external object.

The FIG. 4 is an embodiment of the photocatalytic device of the present disclosure where the carrier is in the form of a plastic food wrap 401 and the external solid object is a food item 402. The FIG. 4 shows the food item is ready to be wrapped by the carrier (the plastic food wrap). The photocatalytic film is coated on the inside surface of the carrier that will make contact with the food item. When a food item is exposed to air, any airborne microbial particles (bacteria, viruses, funguses) may land on the food item. When wrapping the food object with a regular plastic food wrap (without photocatalytic film), the bacteria, virus, and fungus on the surface of the food item could still grow. When wrapping the food object with a plastic food wrap coated with photocatalytic film, the photocatalyst can effectively inhibit the growth of bacteria, virus, and fungus on the surface of the food item, thus preventing bacteria and virus from growing and transmitting to the person consuming the food item, and preventing the food item from becoming stale by inhibiting the growth of the fungus. The photocatalytic film coated on the carrier may be photocatalytic activated by visible light with wavelength>400 nm. No UV light is required. The food item can be of any shape, and it can be raw or cooked or baked. This food preserving application by using a photocatalyst-coated food wrap is not taught by the prior art technique either implicitly or explicitly. In fact, photocatalytic coated food wrap has not existed for the last twenty years even though both photocatalyst and the plastic food wrap are available on the market. It may not be that obvious at all for making a photocatalytic coated plastic food wrap. In some medical applications, it is foreseeable to use a plastic wrap or a plastic bag that is coated with photocatalytic film on both surfaces of the wrap or bag for better antibacterial protection.

In both applications of plastic food wrap and plastic bag, the light shines through the carrier (the plastic food wrap or the bag) first, then shines through the photocatalytic film, and then finally reaching the food item or medical object in the wrap or bag. This is different from the prior art technique that the light shines through the photocatalytic film first and then the carrier. The prior art technique did not teach implicitly or explicitly the use of his photocatalytic device in such a way.

### ADDITIONAL AND ALTERNATIVE IMPLEMENTATION NOTES

Although the techniques have been described in language specific to certain applications, it is to be understood that the appended claims are not necessarily limited to the specific features or applications described herein. Rather, the specific features and examples are disclosed as non-limiting exemplary forms of implementing such techniques.

As used in this application, the term “or” is intended to mean an inclusive “or” rather than an exclusive “or.” That is, unless specified otherwise or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more,” unless specified otherwise or clear from context to be directed to a singular form.

What is claimed is:

1. A photocatalytic device, comprising:
a transparent or translucent carrier; and
at least one photocatalytic film, wherein:
the carrier is in a form of a sheet with two opposite surfaces,
at least one of the two surfaces of the carrier is at least partially coated with the photocatalytic film,
the photocatalytic film is photocatalytic activated by visible light with wavelength greater than 400 nm,
the carrier has an attaching mechanism capable of attaching to an external solid object, and
the carrier is attachable to and detachable from the external solid object with hands of a user without using a tool.

2. The photocatalytic device of claim 1, wherein the carrier is made of a flexible material or a rigid material.

3. The photocatalytic device of claim 2, wherein the flexible material comprises plastics, and wherein the rigid material comprises glass.

4. The photocatalytic device of claim 1, wherein the photocatalytic film contains rhombus-shape anatase-type titanium dioxide (TiO₂).

5. The photocatalytic device of claim 1, wherein the carrier is tinted to filter light in a predetermined wavelength range.

6. The photocatalytic device of claim 1, wherein the carrier is scratch-resistant.

7. The photocatalytic device of claim 1, wherein the carrier is shatter-resistant.

8. The photocatalytic device of claim 1, wherein the carrier comprises a heat insulator.

9. The photocatalytic device of claim 1, wherein the carrier comprises a solar cell.

10. The photocatalytic device of claim 1, wherein the attaching mechanism comprises an adhesive at least partially coated on a first surface of the two surfaces of the carrier to adhere the first surface of the carrier to the external solid object.

11. The photocatalytic device of claim 1, wherein the attaching mechanism comprises electrostatics between the carrier and the external solid object.

12. The photocatalytic device of claim 1, wherein the attaching mechanism comprises a weight of the carrier.

13. The photocatalytic device of claim 1, wherein the attaching mechanism comprises an elastic wrap around an edge of the carrier over the external solid object.

14. The photocatalytic device of claim 1, wherein the attaching mechanism comprises a Velcro between the carrier and the external solid object.

15. The photocatalytic device of claim 1, wherein the attaching mechanism comprises a physical or mechanical locking mechanism that locks the carrier to the external solid object.

16. The photocatalytic device of claim 1, wherein the attaching mechanism comprises wrapping of the carrier over the external solid object.

17. The photocatalytic device of claim 1, wherein the attaching mechanism involves having the carrier in a form of a bag that contains the external solid object.
